(19) United States
(12) Patent Application Publication
(10) Pub. No.: US 2023/0040540 A1
(43) Pub. Date: Feb. 9, 2023

(54) Optimizing Sensor Pressure in Blood Pressure Measurements Using a Wearable Device

(71) Applicant: ChroniSense Medical Ltd., Yokneam (IL)

(72) Inventor: Daniel H. Lange, Kfar Vradim (IL)

(21) Appl. No.: 17/968,663

(22) Filed: Oct. 18, 2022

Related U.S. Application Data
(63) Continuation-in-part of application No. 17/463,284, filed on Aug. 31, 2021.
Application No. 17/463,284 is a continuation-in-part of application No. 15/226,881, filed on Aug. 2, 2016, now Pat. No. 11,160,461.
Application No. 15/226,881 is a continuation-in-part of application No. 14/738,666, filed on Jun. 12, 2015, now Pat. No. 11,160,459.
Application No. 14/738,666 is a continuation-in-part of application No. 14/738,636, filed on Jun. 12, 2015.
Application No. 14/738,636 is a continuation-in-part of application No. 14/738,711, filed on Jun. 12, 2015, now Pat. No. 10,470,692.

(51) Int. Cl. A61B 5/021; A61B 5/024; A61B 5/00; A61B 5/022

(52) U.S. Cl. CPC A61B 5/02108; A61B 5/02416; A61B 5/681; A61B 5/022

(57) ABSTRACT

Systems and methods for optimizing sensor pressure in blood pressure (BP) measurements using a wearable device are provided. An example method includes recording substantially synchronously photoplethysmogram (PPG) data using a PPG sensor and pressure data using at least one pressure sensor on a wearable device, the wearable device having the PPG sensor, and wherein the at least one pressure sensor is substantially located over a user wrist radial artery while an external force gradually applies and releases pressure a plurality of times to the wearable device, wherein the external force is applied to the radial artery. The PPG data and the pressure data is monitored as the PPG data changes in response to the external force being applied and released multiple times during a period. From the recorded PPG and pressure data, a set data us formed of PPG peak values and pressure values. A curve is fitted through the data set. From the curves apex value a MAP value is determined.

### CROSS-REFERENCE TO RELATED APPLICATIONS

The present application is a continuation-in-part of U.S. patent application Ser. No. 17/463,284, titled “Blood Pressure Measurement Using a Wearable Device,” filed on Aug. 31, 2021. The application Ser. No. 17/463,284 is a continuation-in-part of U.S. patent application Ser. No. 15/226,881, titled “Blood Pressure Measurement Using a Wearable Device,” filed on Aug. 2, 2016, now U.S. Pat. No. 11,160,461. The application Ser. No. 15/226,881 is a continuation-in-part of U.S. patent application Ser. No. 14/738,666, titled “Monitoring Health Status of People Suffering from Chronic Diseases,” filed on Jun. 12, 2015, now U.S. Pat. No. 11,160,459, and is a continuation-in-part of U.S. patent application Ser. No. 14/738,636, titled “Wearable Device Electrocardiogram,” filed on Jun. 12, 2015, and is also a continuation-in-part of U.S. patent application Ser. No. 14/738,711, titled “System for Performing Pulse Oximetry,” filed on Jun. 12, 2015, now U.S. Pat. No. 10,470,692. The disclosures of the aforementioned applications are incorporated herein by reference for all purposes, including all references cited therein.

### FIELD

The present application relates to systems and methods for monitoring the health status of people, and more specifically to systems and methods for optimizing the measurement of the diastolic and systolic blood pressure in continuous or intermittent non-invasive blood pressure (NIBP) measurements using wearable devices.

### BACKGROUND

It should not be assumed that any of the approaches described in this section qualify as prior art merely by virtue of their inclusion in this section.

Blood pressure (BP) is one of the basic medical parameters used to diagnose human health condition. The most accurate methods for BP measurements involve insertion of a catheter into a human artery. However, the BP measurements using a catheter are invasive and costly since they require a medical professional to perform the measurements and, typically, can only be performed in a medical facility environment.

Less accurate methods for BP measurements include use of an inflatable cuff to pressurize a blood artery. There are numerous cuff-based portable devices for BP measurements that patients can use at home and do not require assistance of a medical professional. However, cuff-based measurements require inflation and deflation of the inflatable cuff. Therefore, such devices are cumbersome to use and not suitable for ongoing BP measurements.

Some cuff-less devices for BP measurements use an electrical sensor to measure an electrocardiogram (ECG) and optical sensors to measure a photoplethysmogram (PPG). A photoplethysmogram (PPG) is an optically obtained plethysmogram that can be used to detect blood volume changes in the microvascular bed of tissue or in a radial wrist artery. A PPG is often obtained by using a pulse oximeter which illuminates the skin and measures changes in light absorption. The ECG and PPG can be analyzed to determine pulse transit time (PTT). Because the PTT is in-part inversely proportional to the BP, the BP can in some cases be determined from the PTT using a pre-defined relationship. However, changes in a cardio-vascular status of a patient require often re-calibration of PTT based blood pressure measurements. Cuff-less devices can potentially provide continuous monitoring of the BP while imposing a minimal burden on normal activities when worn on various body parts such as a finger, a wrist, or ankle.

Determining the BP based on the PTT alone may not be sufficiently accurate because of other cardiovascular parameters affecting hemodynamics such as vascular resistance, cardiac output, pulse rate (PR), temperature of a finger (if PPG is measured at the finger), and so forth. To compensate for influences of other parameters, some existing techniques for measuring of BP using the PPG include applying correction factors to account for the vascular resistance and age of patient. The correction factors can be determined by an empirical formula. Some other techniques attempt to determine compensation factors to compensate for various additional influences (for example, contacting force to sensors, nervous activity and cardiac output of patient, and ambient temperature). The compensation factors can be determined using a calibration process.

However, all currently known methods for cuff-less, non-inflatable BP or NIBP monitoring require frequent re-calibration to compensate for unaccounted changes in the cardiovascular status of a patient. Moreover, in the PTT and BP measurements carried out using wearable devices, the accuracy of the PTT and BP depends on the pressure that sensors of the wearable device apply to the skin of patient and location of the sensors with respect to blood vessels of a patient. Because the pressure and the location of the sensors change each time the patient puts the wearable device on or corrects location of the wearable device on their body, the corresponding re-calibration would be also required to account for change in the pressure and the location of the sensors. Therefore, there is a need for an NIBP monitoring that can account for changes in the pressure and location of the sensors without frequent re-calibrations. Alternatively, it would be beneficial to have a simple user implemented recalibration method.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to one aspect of the present disclosure, systems and methods for making estimated diastolic and systolic blood pressure measurements with a wearable device are provided. An example method may include recording, by at least one processor, photoplethysmogram (PPG) data using a PPG sensor and pressure data using at least one pressure sensor on a wearable device, while an external force gradually applies and releases pressure a plurality of times to the wearable device. The external force is applied substantially over and perpendicular to the radial artery and can be applied by the wearer of the wearable device. Though the same concepts are applicable to arteries other than the radial artery.

The method includes monitoring, by the processor, PPG data and the pressure data as the PPG data changes in response to the external force being applied and released a plurality of times over a time period. The external force can be the person wearing the device pushing on the wearable device or another person pushing on the device or a mechanism for applying pressure including an automated mechanism. Also contemplated are mechanisms to assist a person in applying pressure where the pressure is applied both by a person and a mechanism for applying pressure.

From the recorded PPG data and pressure data in the period, a set of PPG values corresponding to the PPG pulses and the associated asserted pressure values are extracted from the recorded data. This data set will be used to generate a typically asymmetrical bell curve which is used to estimate a pressure at which PPG pulses are first detected, an apex pressure, and an upper pressure where PPG pulses are no longer detected. These pressures correspond respectively to the diastolic, mean arterial pressure (MAP), and systolic pressure.

Because of the limited number of heart pulses in a period, there is uncertainty in the pressure at which the PPG pulses start and stop. The MAP is likely the most accurate estimate. Thus, the estimated diastolic and systolic values are refined by modifying these values to be consistent with the apex pressure which is the MAP.

The pressure can be increased by the user gradually applying an external pressure to the PPG sensor. The wearable device may include an alarm unit configured to prompt the user to stop applying the external pressure after the pulsating parameter has passed the critical value. The alarm unit may include a haptic device. The alarm unit may include a sound generating device.

According to another example embodiment of the present disclosure, the steps of the method for optimizing sensor pressure in blood pressure measurements using a wearable device are stored on a non-transitory machine-readable medium comprising instructions, which when implemented by one or more processors perform the recited steps.

Other example embodiments of the disclosure and aspects will become apparent from the following description taken in conjunction with the following drawings.

### BRIEF DESCRIPTION OF THE DRAWINGS

Embodiments are illustrated by way of example and not limitation in the figures of the accompanying drawings, in which like references indicate similar elements.

FIG. 1 is a block diagram showing an example system for performing a blood pressure measurement using a wearable device.

FIG. 2 is a block diagram showing components of an example device for performing blood pressure measurement.

FIG. 3 is block diagram illustrating an example device for measuring arterial blood pressure at a wrist.

FIG. 4 shows an example plot of an ECG and an example plot of a PPG.

FIG. 5 shows an example plot of a PPG and an example plot of a blood vessel diameter.

FIG. 6 is a flow chart showing an example method for performing blood pressure measurements.

FIG. 7 shows a diagrammatic representation of a computing device for a machine, within which a set of instructions for causing the machine to perform any one or more of the methodologies discussed herein can be executed.

FIG. 8A is diagrammatic representation of a blood vessel and optical sensor(s).

FIG. 8B is a plot of a compliance of a blood vessel, according to an example embodiment.

FIG. 9 shows plots of PPGs measured at different values of a sensor pressure, according to an example embodiment.

FIG. 10 is a flow chart showing an example method for optimizing sensor pressure in blood pressure measurements.

FIG. 11 is a flow chart showing a method for generating diastolic and systolic blood pressure measurements.

FIG. 12 shows a plot of PPG and MAP measurements and a curve fitted though an estimated MAP point and estimated diastolic and systolic pressure points.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical and electrical changes can be made without departing from the scope of what is claimed. The following detailed description is, therefore, not to be taken in a limiting sense, and the scope is defined by the appended claims and their equivalents.

The present disclosure provides systems and methods for performing BP measurement. Embodiments of the present disclosure allow for continuous or intermittent measuring of blood pressure of a patient in a non-intrusive manner while, for example, the patient is at home, at work, outdoors, traveling, or located at some other stationary or mobile environment. Embodiments of the present disclosure include a wearable device. The wearable device can be worn at the wrist, ankle, chest, neck, or positioned at other sites on a human body. The wearable device can allow measuring blood pressure of the patient without requiring the patient to take an active role in the process. The blood pressure data collected over an extended period of time can be analyzed to detect and track trends in medical parameters and to make conclusions concerning symptoms and progression of one or more chronic diseases from which the patient may suffer.

Some embodiments of the present disclosure can allow optimizing sensor pressure in BP measurements to increase accuracy of determination of the BP. According to some example embodiments, method for optimizing sensor pressure in BP measurements using a wearable deice may include recording, by at least one processor, photoplethysmogram (PPG) data using a PPG sensor of a wearable device while a pressure applied by the PPG sensor to a blood artery of a user is gradually increasing. The method may include monitoring, by the processor, a pulsating parameter associated with the PPG data. The pulsating parameter may change in response to the gradually increasing pressure. The method may include determining, by the processor, that the pulsating parameter has passed a critical value. In response to the determination, the method may include causing, by the processor, the increase of the pressure to stop. The method may include recording, by the processor, further PPG data using the PPG sensor and electrocardiogram (ECG) data using input plates of the wearable device. The method may include analyzing, by the processor, the further PPG data and the ECG data to determine a pulse transit time (PTT), a pulse rate (PR), and a diameter parameter. The diameter parameter may include a change in the diameter of the blood artery. The method may include determining, by the at least one processor and using a pre-defined model, a BP based on the PTT, the PR, and the diameter parameter. The pre-defined model can establish a relationship between the PTT, the PR, the diameter parameter, and the BP.

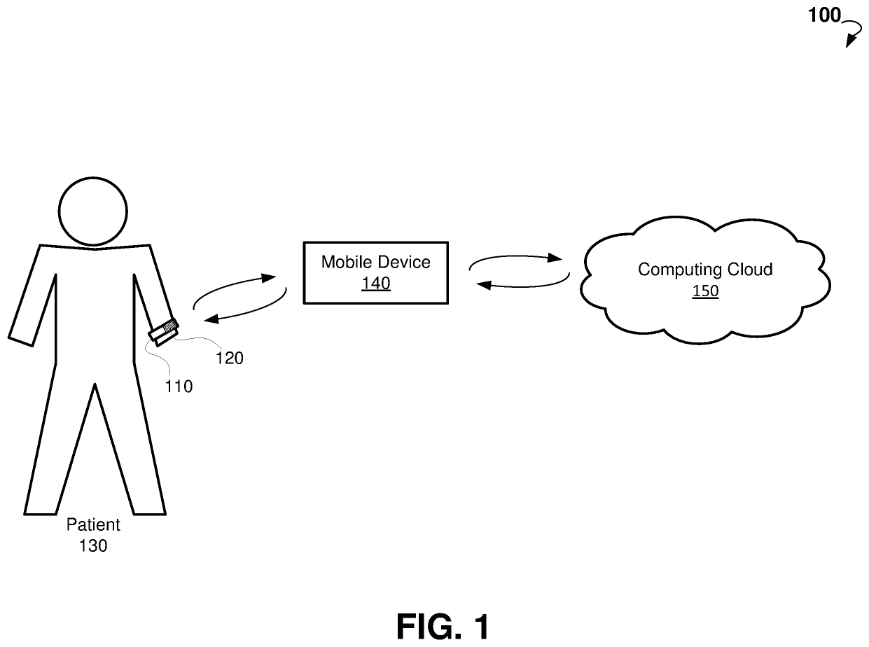

Referring now to FIG. 1, an example system 100 for performing blood pressure measurements is shown. The system 100 can include at least a wearable device 110. The wearable device 110 can include sensors 120. In some embodiments, the wearable device 110 is worn by a patient 130 (for example, on a wrist, ankle, earlobe, neck, chest, fingertip, and the like) for an extended period of time. In various embodiments, the wearable device 110 can be carried out as a watch, a bracelet, a wristband, a belt, a neck band, and the like.

The wearable device 110 can be operable to constantly collect, via sensors 120, sensor data from a patient 130. Based on the sensor data, the wearable device 110 can be operable to provide PPG, ECG and contact pressure data regarding the device 110 against the patient 130. The PPG and ECG can be further used to obtain further medical parameters (for example, pulse rate, pulse transition time, blood pressure, and so forth). The pressure data can be used to determine medical parameters including but not limited to blood pressure.

In some embodiments, the system 100 includes a mobile device 140. The mobile device 140 can be communicatively coupled to the wearable device 110. In various embodiments, the mobile device 140 is operable to communicate with the wearable device 110 via a wireless connection such as, for example, Wi-Fi, Bluetooth, Infrared (IR), or any other wireless or wired communication technology. The mobile device 140 can include a mobile phone, a smart phone, a phablet, a tablet computer, a notebook, and so forth. The mobile device 140 can be operable to receive the sensor data and analyze the sensor data to provide ECG and PPG. Further, the contact pressure

In further embodiments, the system 100 may include a cloud-based computing resource (also referred to as a computing cloud) 150. In some embodiments, the computing cloud 150 includes one or more server farms/clusters comprising a collection of computer servers and is co-located with network switches and/or routers. In certain embodiments, the mobile device 140 is communicatively coupled to the computing cloud 150. The mobile device 140 can be operable to send the sensor data to the computing cloud 150 for further analysis (for example, for extracting medical parameters from the ECG and PPG and storing the results). The computing cloud 150 can be operable to run one or more applications and to provide reports regarding a health status of the patient, based on trends in medical parameters over time.

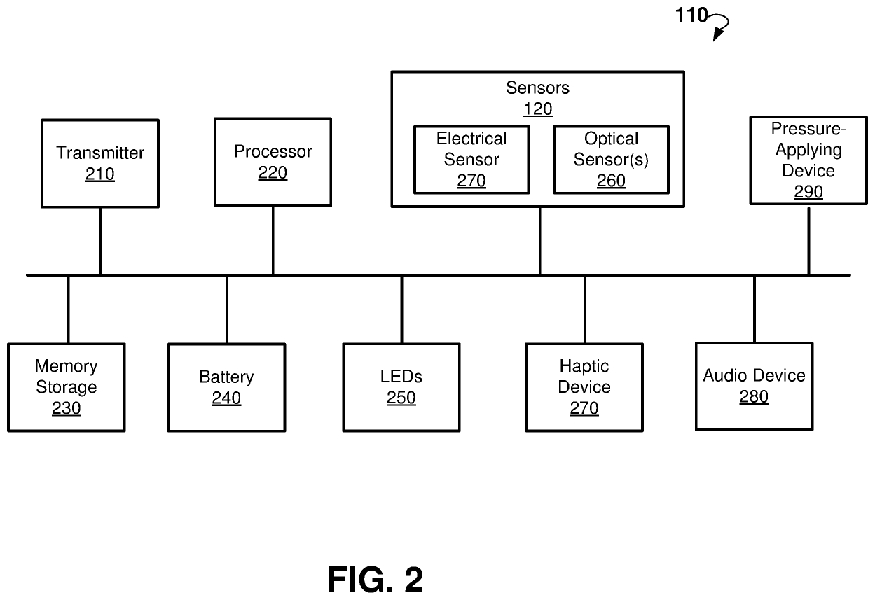

FIG. 2 is a block diagram illustrating components of wearable device 110, according to an example embodiment. The example wearable device 110 includes a transmitter 210, a processor 220, memory storage 230, a battery 240, light-emitting diodes (LEDs) 250, optical sensor(s) 260, electrical sensor 270, a haptic device 270, an audio device 280, and a pressure-applying device 290. The wearable device 110 may comprise additional or different components to provide a particular operation or functionality. Similarly, in other embodiments, the wearable device 110 includes fewer components that perform similar or equivalent functions to those depicted in FIG. 2.

The transmitter 210 can be configured to communicate with a network such as the Internet, a Wide Area Network (WAN), a Local Area Network (LAN), a cellular network, and so forth, to send data streams (for example sensor data, PPG data, and messages).

The processor 220 can include hardware and/or software, which is operable to execute computer programs stored in memory 230. The processor 220 can use floating point operations, complex operations, and other operations, including processing and analyzing data obtained from electrical sensor 270 and optical sensor(s) 260.

In some embodiments, the battery 240 is operable to provide electrical power for operation of other components of the wearable device 110. In some embodiments, the battery 240 is a rechargeable battery. In certain embodiments, the battery 240 is recharged using an inductive charging technology.

In various embodiments, the LEDs 250 are operable to emit light signals. The light signals can be of a red wavelength (typically 660 nm) or infrared wavelength (660 nm). Each of the LEDs 250 is activated separately and accompanied by a “dark” period where neither of the LEDs 250 is on to obtain ambient light levels. In some embodiments, a single LED 250 can be used to emit both the infrared and red light signals. The lights can be absorbed by human blood (mostly by hemoglobin). The oxygenated hemoglobin absorbs more infrared light while deoxygenated hemoglobin absorbs more red light. Oxygenated hemoglobin allows more red light to pass through while deoxygenated hemoglobin allows more infrared light to pass through. In some embodiments of the present disclosure, the LEDs 250 are also operable to emit light signals of isosbestic wavelengths (typically 810 nm and 520 nm). Both oxygenated hemoglobin and deoxygenated hemoglobin absorb the light of the isosbestic wavelengths equally.

The optical sensor(s) 260 (typically a photodiode) can receive light signals modulated by human tissue. Intensity of the modulated light signal represents a PPG. Based on the changes in the intensities of the modulated light signals, one or more medical parameters, such as, for example, oxygen saturation, arterial blood flow, pulse rate, and respiration, can be determined.

The LEDs 250 and optical sensor(s) 260 can be utilized in either a transmission or a reflectance mode for pulse oximetry. In the transmission mode, the LEDs 250 and optical sensor(s) 260 are typically attached or clipped to a translucent body part (e.g., a finger, toe, and earlobe). The LEDs 250 are located on one side of the body part while the optical sensor(s) 260 are located directly on the opposite site. The light passes through the entirety of the body part, from one side to the other, and is thus modulated by the pulsating arterial blood flow. In the reflectance mode, the LEDs 250 and optical sensor(s) 260 are located on the same side of the body part (e.g. a forehead, a finger, and a wrist), and the light is reflected from the skin and underlying near-surface tissues back to the optical sensor(s) 260.

The haptic device 270 can be configured to provide the patient a haptic feedback. For example, the haptic device may include a tap-in device, to apply a force or vibration to skin of the patient.

The audio device 280 can be configured to provide the patient a sound feedback. The audio device 280 can include a beeper configured to generate sounds of one or more pre-determined wavelengths.

The pressure-applying device 290 may be configured to apply external pressure to the optical sensor(s) 260 to force the optical sensor(s) 260 to contact the skin of a patient with different values of a contact force. In some embodiments, the pressure-applying device 290 may include an electrical motor and a spring touching the optical sensor(s) 260. The electrical motor can be configured to stretch the spring gradually causing the optical sensor(s) 260 to apply gradually increasing pressure to the skin of the patient. In other embodiments, the pressure-applying device 290 can include an electrical pump and an inflatable cuff configured to generate external pressure against the optical sensor(s) 260. The electrical pump may inflate the cuff gradually causing the optical sensor(s) 260 to apply gradually increasing pressure to the skin of the patient.

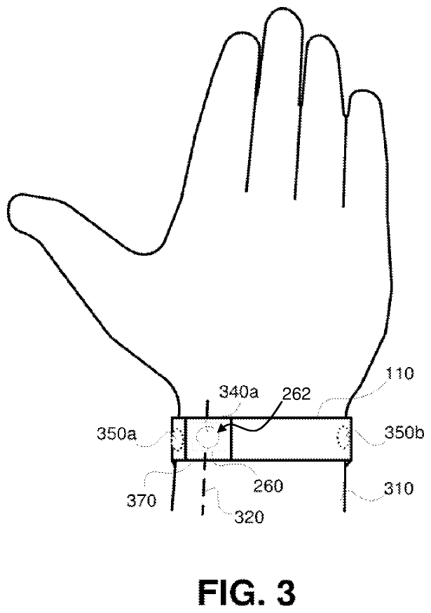

FIG. 3 is a block diagram illustrating an example wearable device 110 placed around a wrist of a patient. In the example of FIG. 3, the wearable device 110 is carried out in a shape of a watch, a ring, and/or a bracelet.

The electrical sensor 270 can include a differential amplifier operable to measure the electrical signal from the wrist. The electrical sensor 370 can include two or more active amplifier input plates embedded in the wearable device at opposite ends. For example, input plates 350a and 350b can be placed in contact with, respectively, the left and right sides of the wrist 310. Alternatively or additionally, two input plates can be placed on opposite sides of the wearable device 110. In some embodiments, the first input plate 340a can be placed on the outer side the wearable device. The second input plate can be placed on the inner side of the wearable device. The second input plate can be in contact with the skin of the patient when the patient wears the wearable device. In some embodiments, the first input plate 340a can be placed in an area 370 of the wearable device 110. The area 270 may cover the radial artery 320 of a patient. The optical sensor(s) 260 can be placed on inner side of the area 370 of the wearable device 110.

In some embodiments, the optical sensor(s) 260 can be placed beneath a pulsating artery travelling along the arm and into a wrist 310. In some embodiments, a radial artery 320 passing in the inner wrist is used for measurements by the optical sensor(s) 260. In other embodiments, other arteries such as the ulnar artery, may be used. An external light source generating constant lighting can be used to radiate the pulsating artery. A beam reflected from the pulsating artery can be intercepted by the optical sensor(s) 260. In certain embodiments, a light of isosbestic wavelength is used to radiate the pulsating artery.

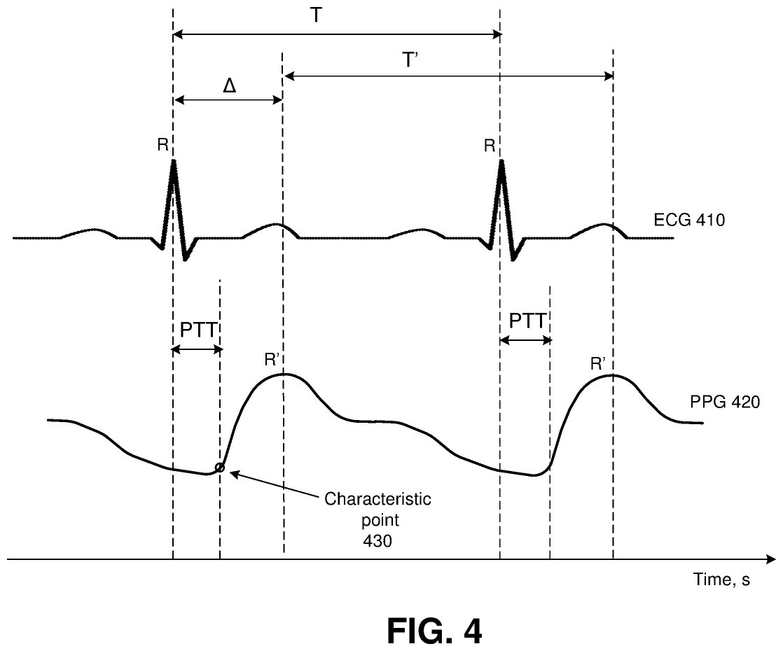

FIG. 4 shows plots of an example an example plot of an ECG 410, and an example plot of a PPG 420. The ECG 410 can be recorded with electrical sensor 270 using input plates placed on the wearable device 110. The ECG 410 can include R peaks corresponding to heart beats. Taking measurements from a single hand or a single wrist is challenging because the difference in voltages between measured locations is miniscule. The electrical signal measured at the wrist can include an ECG 410 and a noise. The noise can be caused by muscle activity, patient movements, and so forth. The noise component can be larger than the ECG. In some embodiments, the signal-to-noise ratio (SNR) is in the range of −40 dB to −60 dB. An example method for measuring a “clean” ECG from a wrist is described in U.S. patent application Ser. No. 14/738,666, titled “Wearable Device Electrocardiogram,” filed on Jun. 12, 2015.

The PPG 420 can be obtained by sensing a change in the color of skin. The change of the skin color is caused by a blood flow in a pulsating artery. In some embodiments, the PPG 420 can include peaks R′ related to the heart beats. Since it takes a time for blood to flow from the heart to the wrist, the peaks R′ are shifted by time periods A relative to the heart beats R in ECG 420. In some embodiments, shifts A can be measured as shift of a waveform of PPG (complex of PPG corresponding to period T′ in FIG. 4) relative to a waveform of ECG (complex of ECG corresponding to period T in FIG. 4).

In various embodiments, ECG 410 and PPG 420 are used to estimate a PTT. In some embodiments, PTT is defined as a time interval between the R peak in ECG 410 and characteristic point 430 located at the bottom of the PPG 420. PTT is a parameter which inversely correlates to BP. PTT decreases as BP increases and PTT increases as BP decreases. Therefore, PTT can be used to estimate BP. In some embodiments, a regression equation can be derived to establish a relation between PTT and BP. The regression equation can be established for both systolic BP and diastolic BP. Alternatively in other embodiments, other mathematical models, such as neural networks, may be used to establish the relation between the PTT and BP.

The location of characteristic point 430 can be uncertain or hard to detect. For example, a shape of PPG at a foot can be diffused when a pulse rate is high. Therefore, in some embodiments, when location of characteristic point 430 is uncertain or hard to detect, shifts between specific features of the ECG and PPG (such as certain landmarks or peaks) corresponding to the same heartbeat are used as an estimate for PTT. In certain embodiments, PTT is estimated based on shifts between waveforms of ECG and PPG corresponding to the same heartbeat.

PTT depends on the shape and cross-section area of a blood vessel (for example, a pulsating artery at which measurement is performed) since speed of blood travelling through the blood vessel depends on the cross-section area of the blood vessel and blood pressure.

According to various embodiments of the present disclosure, ECG and PPG are used to estimate PTT, PR, and diameter of the blood vessel or a change in the diameter of the blood vessel. In some embodiments, PTT, PR, and the diameter of the blood vessel or the change in the diameter of the blood vessel are then used to estimate BP. In some embodiments, PTT is determined based on ECG and PPG. PR can be found using a time period between two consecutive peaks in ECG or two consecutive peaks in PPG. In some embodiments, the diameter of the blood vessel or the change in the diameter of the blood vessel can be estimated using PPG.

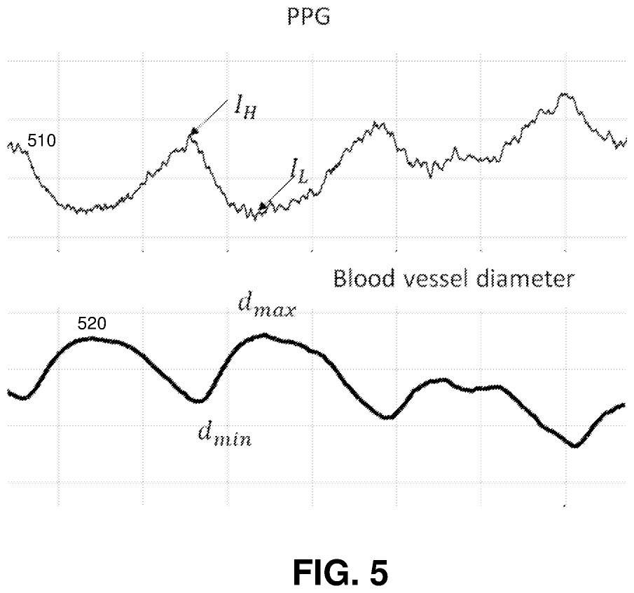

FIG. 5 shows an example plot of PPG 510 and an example plot of blood vessel diameter 520. The PPG 510 represents the intensity I of the light signal as modulated by a human tissue mostly due to a blood flow in the blood vessel. The high peaks (maximums) I_{H} of PPG 510 correspond to the low peaks dₘᵢₙ of the blood vessel diameter 520, and the low peaks I_{L} of the PPG 510 correspond to the high peaks dₘₐₓ of the blood vessel diameter 520.

In some embodiments, the detected PPG signal I, which is the intensity of light signal reflected from pulsating tissue, is modeled as follows:

I(t)=I₀*F*e^{-c*d(t)} (1).

In formula (1), I₀ represents an incident light intensity, F is indicates the absorption by pulsatile tissue, d(t) represents (arterial) blood vessel diameter, and c is overall absorption coefficient of blood hemoglobin derived from a mixture of both oxygen-saturated and non-oxygen saturated hemoglobin. Each of oxygen-saturated and non-oxygen saturated hemoglobin has its own particular value of absorption coefficient c for a particular wavelength of emitted light. Therefore, according to some embodiments, a light of isosbestic wavelength is used to radiate the pulsatile tissue allowing absorption coefficient c so it remains constant and independent of SpO2 oxygen saturation. The light absorption at the isosbestic wavelength is independent of SpO2 oxygen saturation because when a light of an isosbestic wavelength is used, the reflection from the oxygenized blood is the same as reflection from the non-oxygenized blood. In some embodiments, the isosbestic wavelength includes a near infrared wavelength 810 nm (NIR) and a green wavelength 520 nm (green). The NIR wavelength is more suitable for deeper vessels as it has deeper penetration while the green wavelength is more suitable for shallow vessels.

As shown in FIG. 5, the blood vessel diameter 520 changes periodically with the rhythm of the heart rate. The low peaks of the blood vessel diameter dₘᵢₙ correspond to the minimums of the absorption of the light by the blood and the high peaks of the light intensity I_{H}. The high peaks of the blood vessel diameter dₘₐₓ correspond to maximum absorption of the light by blood and the lowest peaks of the light intensity I_{L}. In some embodiments, the low peaks of the blood vessel diameter dₘᵢₙ can be considered to be constant as they reflect lowest diastole. The high peaks of the blood vessel diameter dₘₐₓ may vary relatively slowly due to, for example, fluctuations of blood pressure.

In some embodiments, it can be assumed that

I(t)≈I₀*F*(1−c*d(t)) (2).

Denoting further direct current (DC) component of PPG

DC=I₀*F (3)

and alternative current (AC) component

AC=I₀*F*c*d(t) (4),

an equation for determining blood vessel diameter d(t) can be written as:

$$( \frac{AC}{DC} ) = c * d ( t ) . \qquad ( 5 )$$

In equation (5), the AC component and DC component are found from PPG and absorption coefficient c is known. In some embodiments, change d(t)ₘₐₓ−d(t)ₘᵢₙ is used to estimate BP.

In other embodiments, BP is calculated from measured PTT, PR, and the diameter of the blood vessel or a change thereof using a pre-defined model. The pre-defined model describes a relationship between PTT, PR, and the diameter of the blood vessel and BP. In some embodiments, the pre-defined model is determined using statistical data collected during a calibration process. During the calibration process, a patient can wear the wearable device 110 to measure PTT, PR, and the diameter of the blood vessel or a change in the diameter of the blood vessel. Simultaneously, BP can be measured using an external device (for example, a conventional device for BP measurement). The calibration can be performed once at first usage of the wearable device 110 by a particular patient, and requires at least a single simultaneous measurement by the wearable device 110 and the external device. In other embodiments, several simultaneous measurements should be made to calibrate the wearable device 110 in a range of blood pressure values. The range of blood pressure values can be achieved by taking measurements at either or all the following: different times (hours of a day), different physical states of a patient, and different emotional states of the patient. Alternatively, lowering or elevating the arm and taking local blood pressure at the wrist with both an external device and the wearable device 110 can provide an effective means for mapping the PTT, PR, and diameter of the blood vessel or a change in the diameter of the blood vessel to a wide range of blood pressure values.

In some embodiments, the pre-defined model includes a three-dimensional model, wherein PTT, PR and the diameter of the blood vessel or a change in the diameter of the blood vessel are explanatory variables and systolic blood pressure is a dependent variable. Similarly, another three-dimensional model can be used to establish mathematical relationships between PTT, PR and diameter of blood vessel or a change in the diameter of the blood vessel as explanatory variables and diastolic blood pressure as a dependent variable.

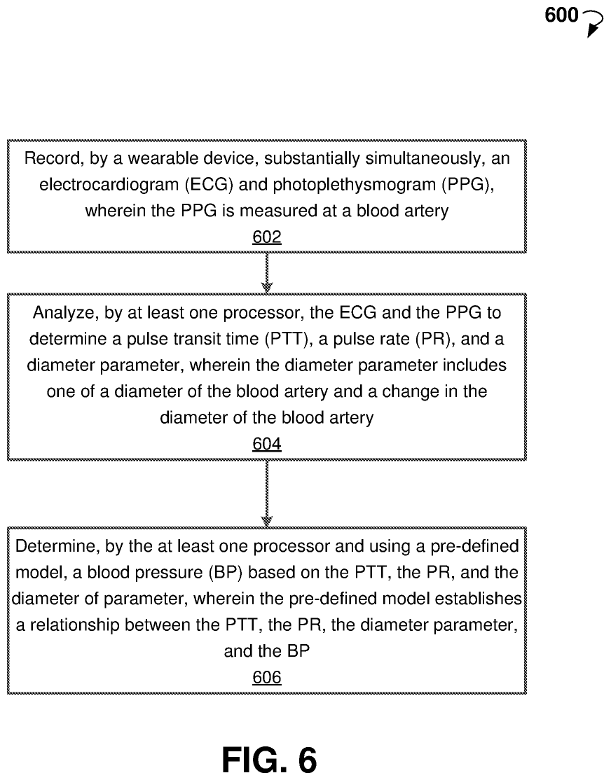

FIG. 6 is a flow chart showing steps of a method 600 for performing BP measurement, according to some embodiments. The method 600 can be implemented using wearable device 110 described in FIGS. 2 and 3 and system 100 described in FIG. 1. The method 600 may commence in block 602 with substantially simultaneous recording, by a wearable device, an ECG and a PPG. In some embodiments, PPG is measured at a blood artery. In some embodiments, ECG and PPG are recorded at a wrist.

In block 604, the method 600 proceeds with analyzing ECG and PPG to determine a PTT, a PR, and a diameter parameter. The diameter parameter may include a diameter of the blood artery or a change in the diameter of the blood artery. In block 606, the method 600 determines, based on PTT, PR, and the diameter parameter, BP using a pre-defined model. The pre-defined model establishes a relationship between the PTT, the PR, the diameter parameter, and the BP. In some embodiments, analysis of ECG and PPG and determination of PTT, the PR, the diameter parameter, and BP is performed locally using processor of the wearable device. In other embodiments, analysis of ECG and PPG and determination of PTT, the PR, the diameter parameter, and BP can be carried out remotely by a mobile device connected to the wearable device or in a computing cloud.

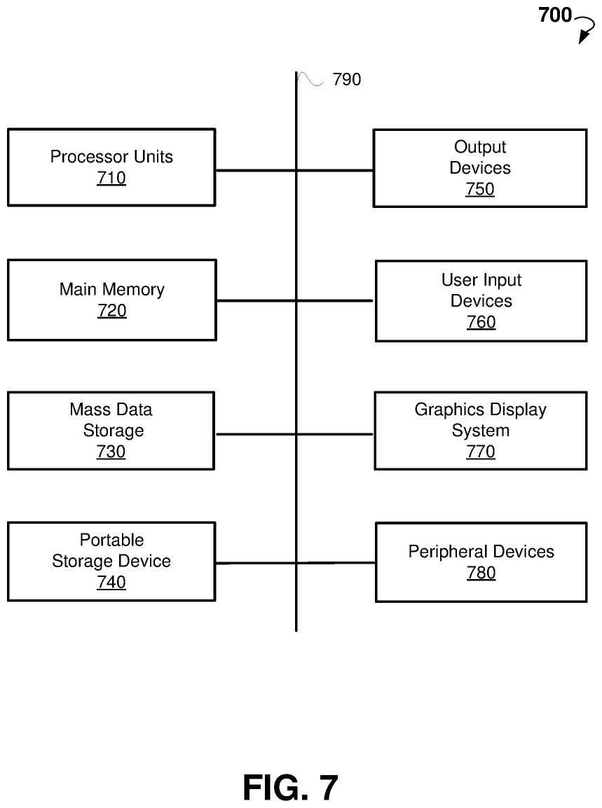

FIG. 7 illustrates a computer system 700 that may be used to implement embodiments of the present disclosure, according to an example embodiment. The computer system 700 may serve as a computing device for a machine, within which a set of instructions for causing the machine to perform any one or more of the methodologies discussed herein can be executed. The computer system 700 can be implemented in the contexts of the likes of computing systems, networks, servers, or combinations thereof. The computer system 700 includes one or more processor units 710 and main memory 720. Main memory 720 stores, in part, instructions and data for execution by processor units 710. Main memory 720 stores the executable code when in operation. The computer system 700 further includes a mass data storage 730, a portable storage device 740, output devices 750, user input devices 760, a graphics display system 770, and peripheral devices 780. The methods may be implemented in software that is cloud-based.

The components shown in FIG. 7 are depicted as being connected via a single bus 790. The components may be connected through one or more data transport means. Processor units 710 and main memory 720 are connected via a local microprocessor bus, and mass data storage 730, peripheral devices 780, the portable storage device 740, and graphics display system 770 are connected via one or more I/O buses.

Mass data storage 730, which can be implemented with a magnetic disk drive, solid state drive, or an optical disk drive, is a non-volatile storage device for storing data and instructions for use by processor units 710. Mass data storage 730 stores the system software for implementing embodiments of the present disclosure for purposes of loading that software into main memory 720.

The portable storage device 740 operates in conjunction with a portable non-volatile storage medium, such as a floppy disk, compact disk (CD), Digital Versatile Disc (DVD), or USB storage device, to input and output data and code to and from the computer system 700. The system software for implementing embodiments of the present disclosure is stored on such a portable medium and input to the computer system 700 via the portable storage device 740.

User input devices 760 provide a portion of a user interface. User input devices 760 include one or more microphones, an alphanumeric keypad, such as a keyboard, for inputting alphanumeric and other information, or a pointing device, such as a mouse, a trackball, stylus, or cursor direction keys. User input devices 760 can also include a touchscreen. Additionally, the computer system 700 includes output devices 750. Suitable output devices include speakers, printers, network interfaces, and monitors.

Graphics display system 770 includes a liquid crystal display or other suitable display device. Graphics display system 770 receives textual and graphical information and processes the information for output to the display device. Peripheral devices 780 may include any type of computer support device to add additional functionality to the computer system.

The components provided in the computer system 700 of FIG. 7 are those typically found in computer systems that may be suitable for use with embodiments of the present disclosure and are intended to represent a broad category of such computer components that are well known in the art. Thus, the computer system 700 can be a personal computer, handheld computing system, telephone, mobile computing system, workstation, tablet, phablet, mobile phone, server, minicomputer, mainframe computer, or any other computing system. The computer may also include different bus configurations, networked platforms, multi-processor platforms, and the like. Various operating systems may be used including UNIX, LINUX, WINDOWS, MAC OS, PALM OS, ANDROID, IOS, QNX, TIZEN and other suitable operating systems.

It is noteworthy that any hardware platform suitable for performing the processing described herein is suitable for use with the embodiments provided herein. Computer-readable storage media refer to any medium or media that participate in providing instructions to a central processing unit, a processor, a microcontroller, or the like. Such media may take forms including, but not limited to, non-volatile and volatile media such as optical or magnetic disks and dynamic memory, respectively. Common forms of computer-readable storage media include a floppy disk, a flexible disk, a hard disk, magnetic tape, any other magnetic storage medium, a CD Read Only Memory disk, DVD, Blu-ray disc, any other optical storage medium, RAM, Programmable Read-Only Memory, Erasable Programmable Read-Only Memory, Electronically Erasable Programmable Read-Only Memory, flash memory, and/or any other memory chip, module, or cartridge.

In some embodiments, the computer system 700 may be implemented as a cloud-based computing environment, such as a virtual machine operating within a computing cloud. In other embodiments, the computer system 700 may itself include a cloud-based computing environment, where the functionalities of the computer system 700 are executed in a distributed fashion. Thus, the computer system 700, when configured as a computing cloud, may include pluralities of computing devices in various forms, as will be described in greater detail below.

In general, a cloud-based computing environment is a resource that typically combines the computational power of a large grouping of processors (such as within web servers) and/or that combines the storage capacity of a large grouping of computer memories or storage devices. Systems that provide cloud-based resources may be utilized exclusively by their owners or such systems may be accessible to outside users who deploy applications within the computing infrastructure to obtain the benefit of large computational or storage resources.

The cloud may be formed, for example, by a network of web servers that comprise a plurality of computing devices, such as the computer system 700, with each server (or at least a plurality thereof) providing processor and/or storage resources. These servers may manage workloads provided by multiple users (e.g., cloud resource customers or other users). Typically, each user places workload demands upon the cloud that vary in real-time, sometimes dramatically. The nature and extent of these variations typically depends on the type of business associated with the user.

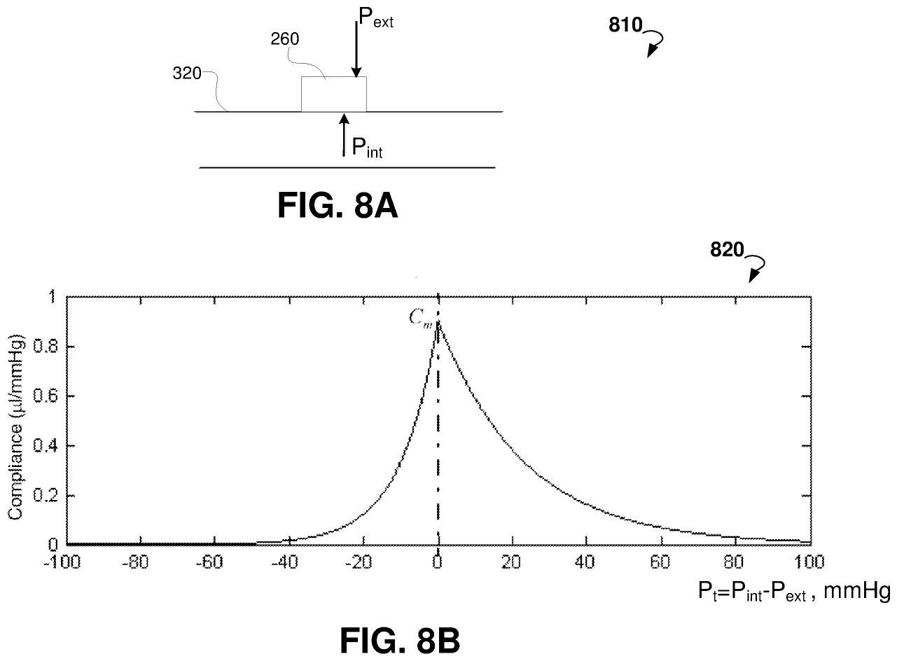

FIG. 8.A is diagrammatic representation of a blood vessel 320 and optical sensor(s) 260. The optical sensor(s) 260 applies pressure Pₑₓₜ to the blood vessel 320. Pᵢₙₜ denotes mean intra-arterial pressure in the blood vessel 320. Determination of the PTT and the BP depend on the accuracy of determination of fluctuation Δd(t) of the blood vessel diameter d(t). The accuracy of determination of fluctuation Δd(t) of the blood vessel diameter d(t) can be contaminated due to either excessive or insufficient amount of the external pressure Pₑₓₜ applied to the blood vessel by the optical sensor(s) 260. Some values of external pressure Pₑₓₜ applied to the blood vessel may result in up to 5% error in PTT and up to 10% in BP.

The fluctuation of the Δd(t) of the blood vessel diameter d(t) depends on the properties of the blood vessel, specifically on compliance C. Compliance C can be determined as follows:

$$C = \frac{Δ V}{Δ P} , \qquad ( 6 )$$

where ΔV is the change of local volume of the blood vessel in response to change ΔP of distending pressure.

FIG. 8B is a plot of compliance C of a blood vessel. The value of the compliance C depends on value of transmural pressure Pₜ. The transmural pressure Pₜ is defined as a difference between the mean intra-arterial pressure Pᵢₙₜ and the external pressure Pₑₓₜ:

Pₜ=Pᵢₙₜ−Pₑₓₜ (7)

As shown in FIG. 8B, the compliance C reaches a maximum value at Pₜ=0. At the maximum value of compliance C the fluctuation of blood vessel volume ΔV (and, correspondently, the fluctuation Δd(t) of the blood vessel diameter d(t)) is maximum. If the external pressure Pₑₓₜ exceeds the mean intra-arterial pressure Pᵢₙₜ or the external pressure Pₑₓₜ is less than the mean intra-arterial pressure Pᵢₙₜ, then the compliance C is not maximum. In these situations, the fluctuation of blood vessel volume ΔV is not maximum.

As shown in FIG. 5, the fluctuation of the PPG 510 correlates with the fluctuation of the blood vessel diameter. Accordingly, at the maximum value of the compliance C, the fluctuation of the PPG 510 is also maximum. This fact can be used to determine a value of sensor pressure corresponding to the maximum value of the compliance C of the blood vessel.

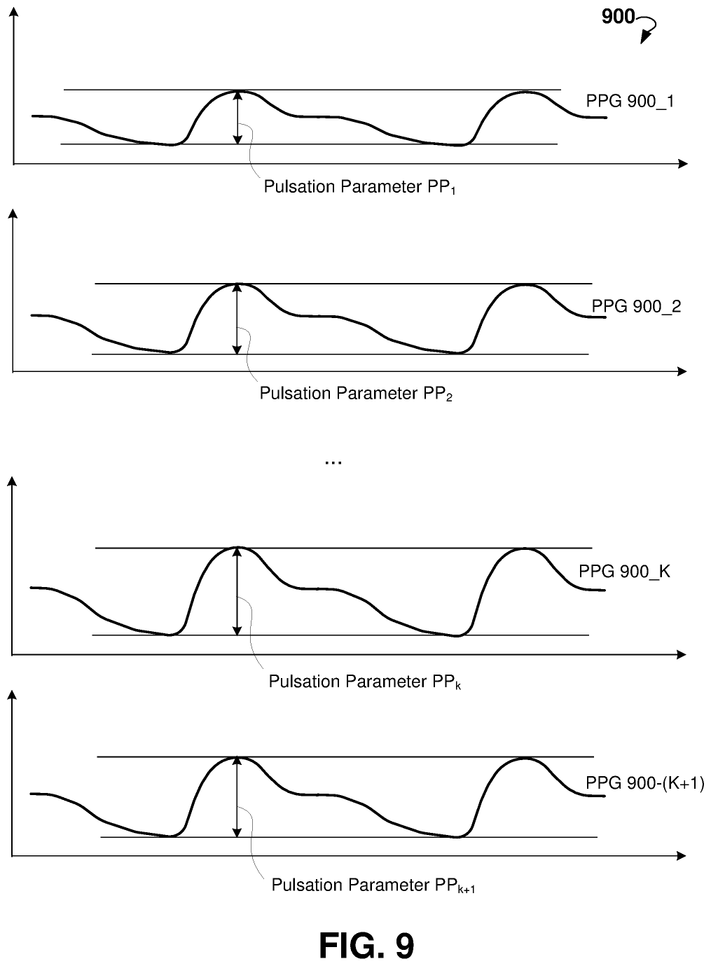

FIG. 9 shows plots of PPGs 900_k measured at different values P_{ext_k} of a pressure of an optical sensor(s) 260, according to some example embodiments. In these embodiments, the different values P_{ext_k} of the pressure applied by the optical sensor(s) 260 to blood vessel can be applied manually by a patient. The patient can be prompted to gradually apply pressure to the optical sensor(s) 260 by using a finger of the other hand at the area 370 of the wearable device 110. As shown in FIG. 3 the area 370 may cover the blood vessel on the wrist of the patient, for example, the radial artery 320. In some embodiment, the patient can be instructed to touch the input plate 340a of the electrical sensor 270 to allow recording two-hand ECG at the same time.

The processor of the wearable device 110 may record, using the optical sensor(s) 260, the PPGs 900_k. For each of the PPGs 900_k, the processor can determine a pulsation parameter PPk. In some embodiments, the pulsation parameter PPk can include a difference between maximums and minimums of the PPGs 900_k. The processor of the wearable device 110 may monitor the change of the pulsation parameter PPk while the pressure P_{ext_k} increases gradually. The processor may determine that the pulsation parameter PPk has passed a critical value, for example, a maximum. For example, the processor may determine that the pulsating parameter has stopped increasing and started decreasing. Correspondently, after the pulsation parameter PPk has passed the critical value, the processor may instruct the patient to stop increasing the pressure on the optical sensor(s) 260. For example, the processor may cause the haptic device 270 to apply a force or vibration to the skin of the patient. Alternatively, the processor may cause the audio device 280 to generate a sound. In some embodiments, the patient may start decreasing the pressure on the optical sensor(s) 260 to allow the pulsating parameter to return to the maximum. The processor may determine that the pulsating parameter has returned to the maximum and instruct the patient to stop decreasing the pressure. For example, the processor may cause the haptic device 270 to vibrate the skin of the patient. The pattern of such vibration can be different from the pattern of the vibration used to prompt the patient to stop increasing the pressure. Alternatively, the processor may cause the audio device 280 to generate a sound. The frequency of the sound can be different from the frequency of the sound used to prompt the patient to stop increasing the pressure.

In other embodiments, the different values P_{ext_k} of the pressure applied by the optical sensor(s) 260 to blood vessel can be created automatically by the pressure-applying device 290. The processor may cause the pressure-applying device 290 to stop the increase in the pressure after the pulsating parameter has passed the critical value. For example, the processor may cause the pressure-applying device 290 to stop the increase in the pressure after determining that the pulsating parameter has stopped increasing and started decreasing, that the pulsating parameter has passed the maximum. The processor may cause the pressure-applying device 290 to decrease the pressure applied by the optical sensor(s) 260 to blood vessel to allow the pulsating parameter to return to the maximum.

After determining that the pulsating parameter has returned to the maximum, the processor may proceed with blood pressure measurements using, for example, method 600 described above with reference to FIG. 6. At these conditions, compliance C of the blood vessel is maximum. Accordingly, the fluctuation of blood vessel volume ΔV and, correspondently, the fluctuation Δd(t) of the blood vessel diameter d(t)) is maximum. Therefore, the errors in estimates of diameter parameter (d(t)ₘₐₓ−d(t)ₘᵢₙ), BP, and PTT are minimum.

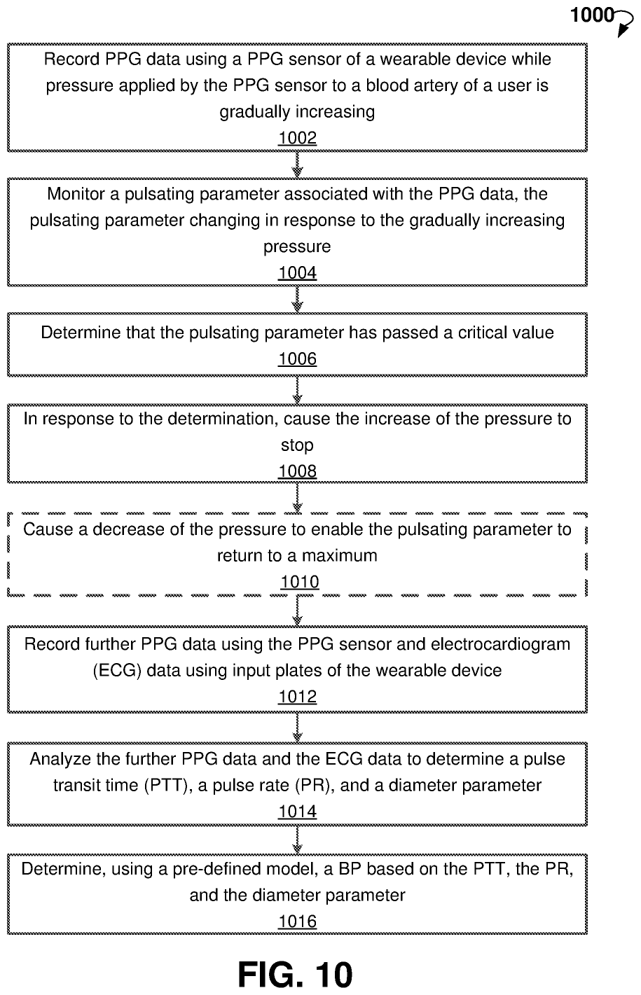

FIG. 10 is a flow chart showing an example method for optimizing sensor pressure in blood pressure measurements, according to some example embodiments. The method 1000 can be implemented using wearable device 110 described with reference to FIGS. 2 and 3 and system 100 described with reference to FIG. 1.

The method 1000 may commence in block 1002 with recording, by at least one processor, PPG) data using a PPG sensor of a wearable device while the pressure applied by the PPG sensor to a blood artery of the user is gradually increasing. The blood artery can be a radial artery of a wrist.

In block 1004, the method 1000 may monitor, by the processor, a pulsating parameter associated with the PPG data. The pulsating parameter may change in response to the gradually increasing pressure and include a difference between a maximum of the PPG data and a minimum of the PPG data.

In block 1006, the method 100 may determine, by the processor, that the pulsating parameter has passed a critical value. The determination that the pulsating parameter has passed the critical value may include determining that the pulsating parameter has stopped increasing and started decreasing. This may indicate that the pulsating parameter has passed the maximum.

The wearable device may include a pressure applying device configured to gradually apply an external pressure to the PPG sensor. Alternatively, the pressure can be increased by the user gradually applying external pressure to the PPG sensor.

In block 1008, in response to the determination that the pulsating parameter has passed the critical value, the method 1000 may stop increasing the pressure. The wearable device may include an alarm unit configured to prompt the user to stop applying the external pressure after the pulsating parameter has passed the critical value. The alarm unit may include a haptic device. The alarm unit may include a sound generating device.

In optional block 1010, the method 1000 may include causing, by processor, a decrease in the pressure to allow the pulsating parameter to return to the maximum. If the pressure is created by the user applying external pressure to the PPG sensor, then the processor may prompt the user to start and stop decreasing the pressure using the alarm unit. If the pressure is created by the pressure applying device, the pressure applying device can decrease the external pressure on the PPG sensor until the pulsating parameter returns to the maximum

In block 1012, the method 1000 may proceed with recording further PPG data using the PPG sensor and electrocardiogram (ECG) data using input plates of the wearable device. In block 1014, the method 1000 may include analyzing, by the processor, further PPG data and ECG data to determine a pulse transit time (PTT), a pulse rate (PR), and a diameter parameter. The diameter parameter may include a change in the diameter of the blood artery.

Determination of the diameter parameter may include modifying the further PPG data by removing, from the further PPG data, an additive contribution resulting from a reflection of a light signal from a surface of a skin covering the blood artery and near-surface tissues underlying the skin and covering the blood artery. During the modification of the PPG data, a contribution resulting from the reflection of the light signal from the blood artery (contribution due to the reflection from the bulk blood volume) is kept unchanged. The additive contribution can be predetermined using a calibration process as described in U.S. patent application Ser. No. 14/738,711, titled “Pulse Oximetry,” filed on Jun. 12, 2015, incorporated herein by reference for all purposes.

The change in the diameter of the blood artery can be determined based on a ratio AC/DC, where AC is an alternating current component of the modified PPG data, and DC is a direct current component of the modified PPG data.

In block 1016, the method 1000 may include determining, by the processor and using a pre-defined model, a BP based on the PTT, the PR, and the diameter parameter. The pre-defined model can establish a relationship between the PTT, the PR, the diameter parameter, and the BP.

### MAP, Diastolic, and Systolic Blood Pressure Estimation

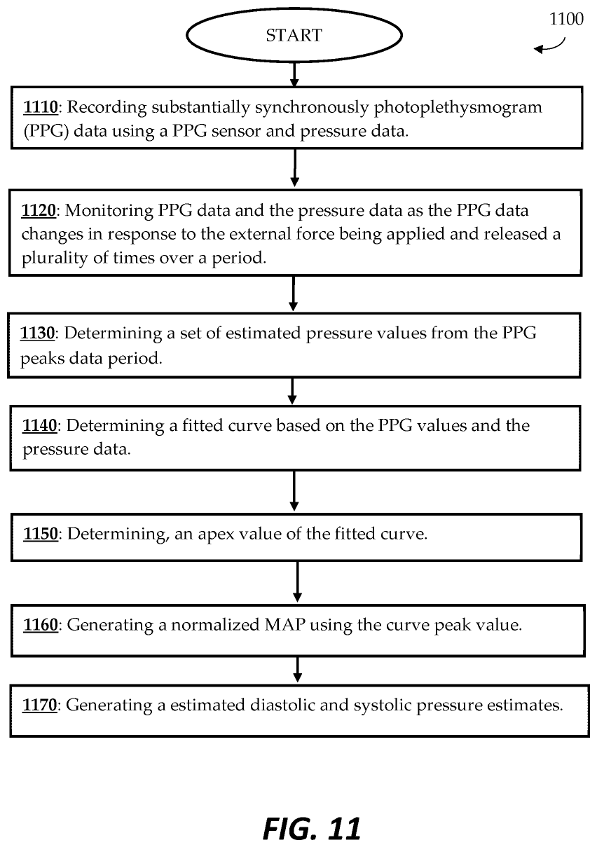

In another novel aspect of the invention, a novel diastolic and systolic blood pressure measurement process using a MAP estimate is shown and disclosed below. This process has several new benefits over the process of FIG. 10 and is disclosed below and shown in FIG. 11. However, this process can be combined with the process of FIG. 10 and thereby providing improved accuracy in blood pressure MAP measurements. First, the new process does not require the collection and processing of ECG data, the process does not require the determination of the PTT, and is less restrictive in the actions that a user needs to make to take a BP measurement. Thus, the new process simplifies the user involvement in the taking of an updated blood pressure measurement.

The process of FIG. 10 based on various physiological characteristic of the PPG signal. The first characteristic is that the PPG signal reaches a maximum when the pressure on the wrist radial artery equals the MAP (Mean Arterial Pressure). The MAP is defined as:

$$M A P = \frac{2 * P d + P s}{3} , \qquad ( 8 )$$

Ps is the systolic BP and Pd diastolic BP. Thus if the pressure asserted against the radial artery is lower than the MAP, the pulse peaks in the PPG sensor signal will occur at a radial pressure value lower than the MAP. As the pressure on the radial artery increases, the PPG signal will increase until a maximum PPG signal is reached. The pressure on the radial artery will equal the MAP. Further pressure by the PPG sensor on the radial artery will cause further restriction of the artery and reduce the pulse peaks in the PPG signal. When sufficient pressure is applied to the artery, the pulses in the PPG signal will be completely suppressed. As pressures against the radial artery is reduced, this process is reversed.

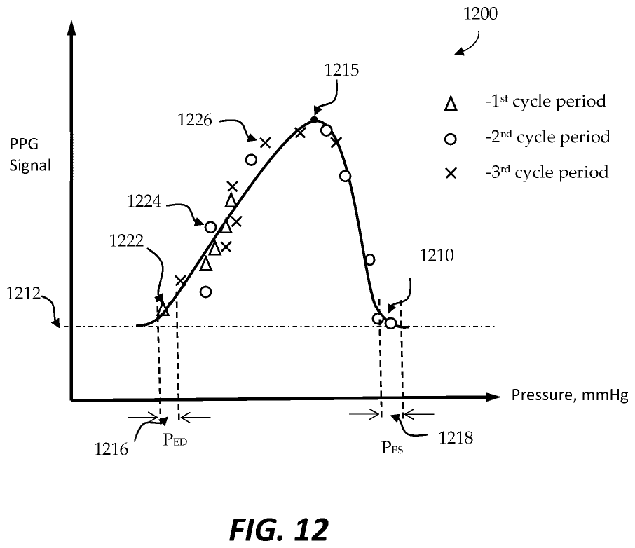

The pressure sensor records the pressure against the radial artery synchronously with the PPG signal. Thus, the plot of the MAP vs. pressure on the radial artery can be represented by an asymmetric “bell” shaped or an asymmetric inverted paraboloid curve where the sensor pressure at the maximum PPG pulse value is the MAP value. The curve is typically asymmetric because of the physiology of the artery. Past testing has found that the curve above the MAP apex is steeper than the curve below the apex. A representative curve of the PPG pulse values vs. Pressure against the radial artery is shown in FIG. 12.

Since it is possible that there is not a pulse peak PPG signal and sensor pressure data point at the MAP peak, the fitted asymetrical curve (asymetrical bell curve) apex is an estimated MAP value.

Another physiological characteristic of the PPG signal and the signal measuring the pressure asserted on the artery is their relationship to the diastolic and systolic pressure in the artery. The pressure asserted against the artery is similar to a blood pressure cuff. The lowest pressure point where the PPG pulse is first detectable, corresponds with a blood pressure cuff, when the cuff pressure is being increased and a heartbeat can be first heard or when the pressure is being released and where the heartbeat can no longer heard or strongly heard. This pressure on the artery corresponds to the diastolic pressure in the artery.

As the pressure against the artery reaches a maximum, the arterial blood flow will be completely cut off and the PPG will not detect a pulse. This is similar to the high pressure in a blood pressure cuff, where the cuff is inflated and increases to the point where the heartbeat is not heard. This is also similar to when to a blood pressure cuff where the pressure is increased beyond where the heart beat is detected and as the cuff pressure is released the heart beat starts being heard. This sensor pressure corresponds to the systolic blood pressure.

Because the number of data points is limited, there is uncertainty in the estimated diastolic and systolic pressures. The PPG pulses may be near the diastolic and systolic pressures but are not likely to be at these exact points. However, estimates from the fitted asymmetrical curve can be used for a range of the estimated diastolic and systolic pressures along with the estimated MAP from the asymmetrical curve, the apex point, to interpolate a more accurate diastolic and systolic blood pressure.

With the estimated diastolic and systolic pressure, there are two equations and two unknowns, the diastolic and systolic pressure. Thus, to generate an improved estimate of the diastolic and systolic BP pressures, the data points at the lower cutoffs (diastolic) and upper cutoffs (systolic) can be assumed to lie in a range around these upper and lower data points.

The process to generate an accurate estimate of the diastolic and systolic blood pressure is to choose a starting point in the range of diastolic and systolic blood pressures and iteratively modify the diastolic and systolic estimates within the range until the iterated estimated diastolic and systolic pressures generate a MAP value that is close to the apex value. The process can pick different starting points on the fitted asymmetric curve and iterated inward, up each side of the asymmetric curve, or outward, down each side of the asymmetric curve. For example, from the data at which the lowest and/or highest pressure at PPG pulses are detected a PPG iteration value is selected above or below this detection value. The amount above or below can be a preset percentage, 10% for example. Next the points where this PPG value intercepts the fitted asymmetric curve is determined, one point on the rising side of the curve and one point on the falling side of the asymmetrical curve. Where a higher PPG pressure is selected, the iteration process will iterate down the diastolic point of the curve and iterate up the systolic estimate. This is reversed if a lower PPG pressure is estimated. Preferably, a higher PPG value is selected.

The literation of the diastolic and systolic pressures can be performed by percentages or fixed amounts. Additionally, the changes to the estimated diastolic and systolic pressures are preferably not by the same amounts or percentages. The asymmetrical curve is steeper for the pressures greater than the apex. Thus, it is contemplated that smaller changes should be made to the estimated systolic pressure.

These ranges in pressure can vary up to 10% from the upper and lower pressure cutoffs however other ranges are contemplated. For example, assuming the MAP apex point indicates the value a MAP of “93” and using the high iteration PPG value, intersects the asymmetric curve with the diastolic pressure of 84 and a systolic pressure of “118”. Note, person with a blood pressure of 120/80 would have a MAP value of 93.3. Using the formula for the MAP, the MAP value would be:

$$M A P = 95.7 = \frac{2 * 84 + 119}{3} . \qquad ( 9 )$$

However, the estimated MAP value of 93 from the curve apex is expected to be the more accurate measurement. Thus, in one embodiment, the diastolic pressure is reduced by an incremental value or a percentage and the systolic value is increased by half this amount. If the iteration amount is “1” for the diastolic and “0.5” for the systolic, then the new estimated diastolic and systolic values are:

$$M A P = 95.2 = \frac{2 * 83 + 119.5}{3} . \qquad ( 10 )$$

In three more iterations of reducing the estimated diastolic pressure by “1” and increasing the systolic pressure “0.5”, the estimated MAP value from the curve based estimated diastolic and estimated systolic pressures are:

$$M A P = 93.5 = \frac{2 * 80 + 120.5}{3} . \qquad ( 11 )$$

As shown in calculation (11) the estimated diastolic and systolic pressure is 80 and 120.5 respectively. Smaller or larger iterations of diastolic and systolic BP can be taken. The ratio of changes of the diastolic to systolic can also vary. For example, a reduction of the diastolic by “1” could be followed by an increase of systolic pressure by “0.4” or “0.6” The ratios can also be a function of the quality of the PPG pulse value data. If there are more consistent data point at the diastolic or systolic points in the fitted asymmetric curve, then the iteration amount may be reduced. Further, the diastolic or systolic pressure estimates can be iterated one at a time with increases or decreases. Alternatively, pressures can be increased or decreased one at a time. This decision can depend on the number of PPG and pressure data points taken in a cycle or multiple cycles. Further, prior readings or history can be used to determine if either the diastolic or systolic pressure reading can be used or which of the diastolic or systolic pressures are more likely in error. For example, if the pressure is applied too quickly to the wrist band, then the number of the PPG peaks and sensor pressure data points may be too course. The data point for where the PPG pulse is no longer detected, the systolic pressure, could occur above the actual systolic point.

When collecting PPG and pressure datapoints, the points where the PPG pulse is first detected when applying pressure may be more accurate than the highest-pressure data point. Though, the opposite could occur. If insufficient pressure is applied, then the pressure where the PPG pulse cut-off occurs may not be achieved. Thus, the PPG and pressure data points need to be evaluated to determine if one or both the diastolic and systolic points can be used. If only one of the diastolic and systolic data points is determined to be sufficiently accurate, then the diastolic or systolic estimate can be used to estimate the other parameter using the MAP. An example of this is shown in equations (12) and (13).

$$M A P = 93 = \frac{2 * 82 + Pes}{3} . \qquad ( 12 )$$

$$Pes = 115 = 3 * 93 - 2 * 82. \qquad ( 13 )$$

FIG. 11 discloses the process 1100 of using a PPG sensor and a pressure sensor for the generation of an estimated diastolic and systolic blood pressure using an estimated MAP pressure value (the apex) from an asymmetrical fitted curve and curve estimated points where the PPG pulses are no longer detected. The PPG component 250, 260 is comprised of LED lights 250 and an optical sensor 260. The optical sensor generates the PPG signal from which the PPG data is generated. The pressure sensor 262 is shown co-located with the PPG sensor 260. However, the pressure sensor 262 can be located near the PPG sensor 260. Further, multiple pressure sensors can be used to provide an average reading if needed. Preferably, the pressure sensor 262 is co-located with the PPG sensor 260 so that the pressure measured by the PPG sensor 260 matches the pressure asserted against the radial artery 320.

The sampling and recording of the PPG data and pressure data is performed substantially synchronously. This data can be stored in the wearable device 110 and transmitted or uploaded to a mobile device 140 that is in communication with the wearable device 110. The PPG and pressure data from the PPG sensor 260 and pressure sensor 262 can be uploaded to the mobile device 140 for processing into a fitted asymmetrical curve where the apex value of the curve is the MAP. This data can be preprocessed for later transmission to the computing cloud 150. This data can also be transmitted to the computing cloud 150 for storage, processing, display and analysis with prior MAP data or BP data acquired by other means including but not limited to a blood pressure cuff.

In step 1120, the beginning of the estimated diastolic and systolic blood pressure estimation process is detected. The process starts when the data from the pressure sensor indicates an increasing external force being applied by the wearable device 110 to the radial artery 320. Alternatively, the calibration process could begin by an indication being output by the wearable device 110 or to the wearer of the device or by a user invoking the calibration through an input to the wearable device 110 or mobile device 140.

The period is defined as multiple cycles of externally applying increased pressure on the wearable device wrist strap and the pressure returning substantially back to the starting value. A cycle is the time from when the external pressure to the wearable device starts to when the external pressure is release or ends. Preferably this time is at least one second, but three to five seconds is better because more PPG pulses corresponding to heart beat are recorded. Longer cycle times are contemplated. However, the longer the cycle the harder it is for a person to execute. Preferably, the cycle is repeated multiple times, and the external pressure is sufficient to reach a peak PPG pulse value and subsequent reduction of the PPG signal as the pressure is increased. At least three cycles are preferable, but more cycles are contemplated by the invention.

In step 1130, a set of PPG peak values associated with the systolic pressure and the pressure data are determine during the period. For each heart pulse, the PPG peak data value are determined, and the associated synchronously recorded pressure data are paired. This provides a set of data points to which an asymmetric bell shaped curve can be fitted.

In step 1140, an asymmetric bell shape curve is fitted based on the set of associated PPG peak values and pressure values. An example of a fitted curve 1210 is shown in FIG. 12 which is discussed in more detail below. The curve 1210 can be an asymmetric bell shaped or an inverted asymmetric paraboloid which can be expressed as a polynomial. A person of ordinary skill in the art of curve fitting data would be able to fit a asymmetric curve to the data set.

There are three important sections of the asymmetric fitted curve. The first section is the apex of the asymmetric curve which corresponds to the MAP value. The second curve section is the lowest asserted pressure where PPG pulses are first detected. This section of the curve applies a range of estimated diastolic blood pressure. The third section of the curve is where the highest asserted pressure where PPG pulses are no longer detected which corresponds to the systolic pressure.

If a cycle of data values are too random or have too much variance, then the invention contemplates not using all or some of the points in a cycle. Additionally, if some measure of the curve fit indicates that the quality of the points in the set are not sufficient to match a curve, additional measures can be taken. The problems might be that the user pressed too fast on the wearable device wrist band and the cycle only included less than a full heart pulse or only a couple of heart pulses. Also, if the user does not press on the wearable device wrist band with sufficient pressure, the apex point will not be reached. This can impede the accuracy of the diastolic and systolic blood pressure estimates.

Data from past calibrations could be used if temporally close. For example, prior readings within the last 5 minute may be used. Additionally, the wearable device may give feedback to repeat the process, repeat the process slower, or repeat the process with more pressure asserted on the wrist band. A reliable calibration may use the data sets from one or more of these calibration processes.

In step 1150, the apex or the top of the curve is determined. It is at this point that the external pressure matches the MAP. This is the pressure that is used in generating an estimated diastolic and systolic blood pressure.

In step 1160, a normalize MAP value is generated. The pressure data from the pressure sensor may need to be scaled and translated to provide a normalized MAP value. In this step the curve's apex pressure value can be scaled and an offset added or subtracted from the curve's apex value.

In step 1170, an estimated diastolic and systolic blood pressure is generated from the PPG and pressure value data points and the pressure sensor data points and using the estimated MAP value. As described above, the initial pressure at which PPG pulses are first detected and the higher pressure at which the PPG pulses are not detectable are extracted from the multiple cycles of pressing on the wrist band. Values around these points are iterated as described above to generate an estimated diastolic and systolic blood pressure with an estimated MAP that matches the apex on the fitted asymmetric curve.

As shown in FIG. 12 is a plot 1200 of the PPG peak values vs. pressure values 1222, 1224, 1226 from three cycles of asserting pressure on the wristband 110 and an asymmetric curve 1210 fitted to these points. The PPG values are taken from the peaks of the PPG signal which corresponds to the systolic phase of a heartbeat. The associated pressure being asserted against the radial artery by the pressure sensor, which corresponds to the pressure asserted by the PPG sensor against the radial artery, form the values for the data set. From this set of PPG/pressure values, an asymetrical curve 1210 is fitted so that the curve apex 1215 can be determined. The pressure value at the curve apex 1215 is the estimated MAP value and should correspond to the true MAP.

The triangular identified values, “A” 1222 show a cycle where the pressure on the wearable device wrist band is not sufficient to exceed the apex pressure point 1215 where the PPG signal starts to decline. While alone, these points 1222 are insufficient to determine a curve 1210 and an apex 1215, they are useful for curve fitting when used in conjunction with data from other cycles.

The circular identified values, “0” 1224 show a cycle where the pressure on the wearable device wrist band exceeds the apex pressure 1215 and reaches the pressure point 1218 where the arterial blood flow is cut off and no systolic peaks in the PPG sensor are detected.

The values identified by a cross “X” 1226 show a cycle where the external pressures exceed the apex 1215 point but the pressure sensor does not reach sufficient arterial pressure to cut off the arterial blood flow.

The pressure PED 1216 is the estimated diastolic blood pressure and is where the systolic peaks in the PPG signal are first detectable. The pressure PES 1218 is the estimated systolic blood pressure where arterial blood flow is cut off. These pressures are shown as a range because of the limited number of heart beats taken during the multiple cycles. This range is selectable and can be up to 10% of the pressure but other ranges are contemplated. Pressures within this range are used as described above with the MAP (apex) to generate improved estimates of the diastolic and systolic blood pressures. The PPG signal value 1212 represents a constant background signal level from tissues around and over the artery.

Thus, methods and systems for optimizing sensor pressure in blood pressure measurements using wearable devices have been described. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A method for optimizing sensor pressure in a blood pressure (BP) measurement, the method comprising:
recording substantially synchronously, by at least one processor, photoplethysmogram (PPG) data using a PPG sensor and pressure data using at least one pressure sensor on a wearable device, the wearable device having the PPG sensor, and wherein the at least one pressure sensor is substantially located over a user wrist radial artery while an external force gradually applies and releases pressure a plurality of times to the wearable device, wherein the external force is applied substantially over and perpendicular to the radial artery;
monitoring, by the at least one processor, PPG data and the pressure data as the PPG data changes in response to the external force being applied and released a plurality of times over a period;
determining, by the at least one processor, from the PPG data and pressure data in the period, a set of PPG pulse values corresponding to the systolic heart pulse phase and the associated pressure values;
determining, by the at least one processor, an asymmetrical bell curve based on the set of PPG pulse values and the associated pressure values;
determining, by the at least one processor, an apex pressure value of the asymmetrical curve;
determining, by the at least one processor, an estimated diastolic pressure and an estimate systolic pressure from the asymmetrical bell curve; and
iterating the estimated diastolic pressure and the estimated systolic pressure until a value calculated using the MAP equation based on the iterated estimated diastolic pressure and the iterated estimated systolic pressure is substantially equal to the apex value, thereby generating an estimated diastolic and estimated systolic blood pressure.

2. The method of claim 1, wherein the asymmetrical bell curve is a paraboloid, and the apex value is the paraboloid vertex.

3. The method of claim 2, wherein the paraboloid is a polynomial equation for each side or the curve.

4. The method of claim 3, wherein the wearable device includes an alarm unit configured to prompt the user to stop applying the external pressure after the pulsating parameter has passed the critical value.

5. The method of claim 1, wherein the external force is applied by a wearer of the wearable device or by an automatic pressure assertion mechanism.

6. The method of claim 5, wherein the plurality of times over a period is at least three times.

7. The method of claim 6, wherein the period is at least six seconds.

8. The method of claim 1, wherein the iteration of the estimated diastolic pressure is by a greater amount than the estimated systolic pressure.

9. The method of claim 8, wherein the estimated systolic pressure is iterated by forty to sixty percent of the estimated diastolic pressure.

10. The method of claim 1, wherein the value is within plus or minus three percent of the apex value.

11. A system for optimizing sensor pressure in a blood pressure (BP) measurement, the system comprising:
a wearable device including a photoplethysmogram (PPG) sensor at least one pressure sensor; and
at least one processor communicatively coupled to the wearable device, the at least one processor being configured to:
record substantially synchronously, by at least one processor, photoplethysmogram (PPG) data using a PPG sensor and pressure data using at least one pressure sensor on a wearable device, the wearable device having the PPG sensor, and wherein the at least one pressure sensor is substantially located over a user wrist radial artery while an external force gradually applies and releases pressure a plurality of times to the wearable device, wherein the external force is applied substantially over and perpendicular to the radial artery;
monitor, by the at least one processor, PPG data and the pressure data as the PPG data changes in response to the external force being applied and released a plurality of times over a period;
determine, by the at least one processor, from the PPG data and pressure data in the period, a set of PPG pulse values corresponding to the systolic heart pulse phase and the associated pressure values;
determine, by the at least one processor, an asymmetrical bell curve based on the set of PPG pulse values and the associated pressure values;
determine, by the at least one processor, an apex pressure value of the asymmetrical curve;
determine, by the at least one processor, an estimated diastolic pressure and an estimate systolic pressure from the asymmetrical bell curve; and
iterate the estimated diastolic pressure and the estimated systolic pressure until a value calculated using the MAP equation based on the iterated estimated diastolic pressure and the iterated estimated systolic pressure is substantially equal to the apex value, thereby generating an estimated diastolic and estimated systolic blood pressure.

12. The system of claim 10, wherein the asymmetrical bell curve is a paraboloid, and the apex value is the paraboloid vertex.

13. The system of claim 12, wherein the paraboloid is a polynomial equation for each side or the curve.

14. The system of claim 13, wherein the wearable device includes an alarm unit configured to prompt the user to stop applying the external pressure after the pulsating parameter has passed the critical value.

15. The system of claim 11, wherein the external force is applied by a wearer of the wearable device or by an automatic pressure assertion mechanism.

16. The system of claim 15, wherein the plurality of times over a period is at least three times.

17. The system of claim 16, wherein the period is at least six seconds.

18. The system of claim 11, wherein the iteration of the estimated diastolic pressure is by a greater amount than the estimated systolic pressure.

19. The system of claim 18, wherein the estimated systolic pressure is iterated by forty to sixty percent of the estimated diastolic pressure.

20. The system of claim 11, wherein the value is within plus or minus three percent of the apex value.

21. A method for optimizing sensor pressure in a blood pressure (BP) measurement, the method comprising:
recording substantially synchronously, by at least one processor, photoplethysmogram (PPG) data using a PPG sensor and pressure data using at least one pressure sensor on a wearable device, the wearable device having the PPG sensor, and wherein the at least one pressure sensor is substantially located over a user wrist radial artery while an external force gradually applies and releases pressure a plurality of times to the wearable device, wherein the external force is applied substantially over and perpendicular to the radial artery;
monitoring, by the at least one processor, PPG data and the pressure data as the PPG data changes in response to the external force, and a pulsating parameter associated with the PPG data, the pulsating parameter changing in response to the external force being applied and being applied and released a plurality of times over a period;
determining, by the at least one processor, from the PPG data and pressure data in the period, a set of PPG pulse values corresponding to the systolic heart pulse phase and the associated pressure values;
determining, by the at least one processor, that the pulsating parameter has passed a critical value;
in response to the determination, causing, by the at least one processor, the increase of the pressure to stop;
recording, by the at least one processor, further PPG data using the PPG sensor and electrocardiogram (ECG) data using input plates of the wearable device;
analyzing, by the at least one processor, the further PPG data and the ECG data to determine a pulse transit time (PTT), a pulse rate (PR), and a diameter parameter, wherein the diameter parameter includes a change in the diameter of the blood artery; and
determining, by the at least one processor and using a pre-defined model, a BP based on the PTT, the PR, and the diameter parameter, wherein the pre-defined model establishes a relationship between the PTT, the PR, the diameter parameter, and the BP, wherein the BP includes and BP diastolic and BP systolic.
determining, by the at least one processor, an asymmetrical bell curve based on the set of PPG pulse values and the associated pressure values;
determining, by the at least one processor, an apex pressure value of the asymmetrical curve;
determining, by the at least one processor, an estimated diastolic pressure and an estimate systolic pressure from the asymmetrical bell curve;
iterating the estimated diastolic pressure and the estimated systolic pressure until a value calculated using the MAP equation based on the iterated estimated diastolic pressure and the iterated estimated systolic pressure is substantially equal to the apex value, thereby generating an estimated diastolic and estimated systolic blood pressure; and
averaging the estimated diastolic blood pressure with the BP diastolic blood pressure and the estimated systolic blood pressure with the BP systolic blood pressure.
